(19) United States
(12) Patent Application Publication
(10) Pub. No.: US 2004/0241166 A1
(43) Pub. Date: Dec. 2, 2004

(54) Use of inhibitors of ikk-beta and method for discovery of said inhibitors

(76) Inventors: Martin Ungerer, Munchen (DE); Korbinian Brand, Munchen (DE); Meinrad Gawaz, Munchen (DE)

Correspondence Address: MYERS BIGEL SIBLEY & SAJOVEC, PO BOX 37428, RALEIGH, NC, 27627, US

(21) Appl. No.: 10/473,011

(22) PCT Filed: Mar. 26, 2002
(86) PCT No.: PCT/EP02/03382

(30) Foreign Application Priority Data
Mar. 27, 2001 (DE) 101150733

(51) Int. Cl. A61K 39/395; A61K 31/00; C12Q 1/68; G01N 33/567

(52) U.S. Cl. 424/146.1; 514/44; 514/1; 435/6; 435/7.21

(57) ABSTRACT

The invention relates to the use of an IKK-β inhibitor for the production of a medicament for the treatment or prevention of atheroscieroses. A screening method for the discovery of IKK-β inhibitors in endothelial cells is also disclosed. The method involves the contacting of a potential inhibitor with activated endothelial cells and a determination of the secretion of MCP-1 from the endothelial cells.

The present invention relates to a new use of specific inhibitors for the preparation of medicaments for the prevention and treatment of atherosclerosis and to a specific screening method for discovering new inhibitors.

It is known that atherosclerosis is a consequence of lesions of the vascular endothelium which are promoted by hypertension, local vortex formation or oxygen deficiency. It is assumed that, owing to the subendothelial structures of the vessel wall which are exposed by the lesions, circulating blood platelets adhere to the damaged sites of the endothelium (platelet adhesion) and then clump together (platelet aggregation).

As a result of the interaction of the blood platelets with the endothelial cells a large number of secondary processes occur which lead to an accumulation of lipids in the vessel wall. With the accumulation of lipids in the vessel wall, the formation of so-called atherosclerotic plaques commences. Foam cells formed from immigrant macrophages accumulate in the plaques. Foam cells absorb the accumulated lipids and degrade them. While some lipo-proteins can be easily degraded, cholesterol and cholesterol esters remain practically uncleaved. The foam cells therefore become overloaded with cholesterol and cholesterol esters.

As a consequence of atherosclerotic plaques, central necroses may be produced, in the vicinity of which calcium salts are deposited. In advanced stages, plaques may tear, producing atherosclerotic lesions that are subsequently covered by a thrombus. Thickening of the arterial wall and the formation of surface thrombi may constrict the vascular lumen to the extent of complete occlusion. Thrombi that become detached from larger arteries cause occlusion of smaller arteries in the periphery of the body. Arterial circulation disorders, which are due in most cases to atherosclerotic processes, are the most common cause of death in the so-called industrialised nations, accounting for approximately 50% of cases.

The molecular mechanisms of atherogenesis are barely understood, particularly with regard to processes that determine the chemotaxis of monocytes and the transmigration of monocytes. It is, however, already known that the cytokine MCP-1 (Monocyte Chemoattractant Protein-1) occurs in inflammatory processes of the arterial wall and attracts monocytes to the site of the inflammation. It is also known that MCP-1 can be expressed in endothelial cells. The expression and secretion of MCP-1 is controlled by way of complex signal cascades. Details of the control of the expression and secretion of MCP-1 in endothelial cells in early stages of atherogenesis are not known from the prior art. In particular, no target proteins the influencing of which can be used to influence the occurrence and the early course of the disease are known in connection with atherosclerosis.

The prevention or lessening of the attraction and immigration of monocytes into the vessel wall in the course of atherosclerotic processes can slow the accumulation of foam cells in atherosclerotic plaques and would therefore be suitable for suppressing or preventing atherosclerotic processes.

It is an object of the invention to provide a new method by which atherosclerosis can be prevented or treated.

It is a further object of the present invention to provide a screening method for discovering new active substances with which atherosclerosis can be prevented or treated.

Those objects are achieved in accordance with the claims. The present invention is founded on the realisation that aggregation of blood platelets on the vascular endothelium is not a pre-requisite for expression of MCP-1 in endothelial cells. Rather, activated blood platelets already induce expression of MCP-1 in endothelial cells at a stage of atherogenesis at which blood platelet aggregation is not yet detectable. It has been found that activated blood platelets, which in vivo, without clumping, adhere firmly but only transiently to an endothelial monolayer activated by ischaemia, degranulate and activate the transcription factor NF-κB in the neighbouring endothelial cells. That new form of interaction between blood platelets and activated endothelial cells, which does not require perceptible lesions, differs on the one hand from the rolling interactions known from the prior art, in which firm adhesion does not occur, and, on the other hand, from the firm adhesion and clumping in the case of platelet aggregation, which is not merely transient but permanent. The activation of NF-κB in the endothelial cells which is mediated by activated blood platelets leads to secretion of MCP-1 from the endothelial cells. As a result of the secretion of MCP-1, monocytes which are able to migrate into the vessel wall are attracted in early stages of atherogenesis. A merely transient adhesion of activated blood platelets leads, therefore, to secretion of MCP-1, which is a pre-requisite for the formation of plaques in atherosclerotic processes.

The present invention is further founded on the realisation that, in the case of activation of NF-κB mediated by activated blood platelets, the inhibitor protein IκB is specifically phosphorylated, and thus activated, by the IKK-β kinase of the IKK complex. The specific short-duration activation by activated blood platelets results, with the participation of IKK-β, in a strong and continuing phosphorylation activity of IκB which lasts for up to one hour. The associated degradation of the inhibitor protein IκB results in a prolonged and strong activation of NF-κB and a corresponding secretion of MCP-1. That is surprising in view of the fact that the interaction with blood platelets takes place for only a comparatively short time.

The time-related activation profile of the IKK complex in the case of activation by activated blood platelets is fundamentally different from that of activation by IL-1β or TNF which are already known from the prior art as activators of the IKK complex. Activated blood platelets activate the IKK complex for approximately 60 minutes, the maximum phosphorylation level being reached after approximately 30 minutes and hardly any difference being observed between the activation of IKK-α and IKK-β. TNF leads to an extremely strong activation, especially of the IKK complex, of only a few minutes duration, which, however, is no longer detectable after about 20 minutes. IL-1β, on the other hand, causes a very prolonged activation, especially of IKK-β, which continues for about two hours. The activation of IKK-β is, however, substantially weaker in that case than activation by activated blood platelets.

By activation of the IKK complex with activated blood platelets, therefore, the kinase IKK-β is activated strongly and for a prolonged period, which is decisive for activation of NF-κB in connection with secretion of MCP-1 and hence for the formation of plaques in atherosclerotic processes. That mechanism concerns a decisive step not only in the regulation of early atherosclerotic processes but also in plaque formation, plaque propagation and plaque destabilisation.

The invention therefore proposes the use of an inhibitor of IKK-β for the preparation of a medicament for the treatment or prevention of atherosclerosis.

The invention offers the advantage that, when IKK-β is inhibited at an early stage of athero-genesis, the endothelial layer is not yet shielded by platelet aggregation or surface thrombus formation and therefore the endothelial cells can be easily and effectively reached by inhibitors that influence expression of MCP-1. That stage is characterised by minimal endothelial lesions, often so-called “fatty streaks”.

The invention further offers the advantage that, when IKK-β is inhibited in endothelial cells neighbouring atherosclerotic plaques, enlargement of the plaques can be prevented or existing plaques can be stabilised.

The invention further offers the advantage that a protein in the signal cascade of the expression of MCP-1 that plays a central role in the case of activation by activated blood platelets is inhibited, that protein not being NF-κB, the direct influencing of which would lead to serious side-effects since NF-κB is also involved in the expression of other proteins, such as in the expression of cytokines, chemokines, growth factors, for example TNF, IL-1β, IL-8, adhesion molecules, for example ICAM-1, VCAM-1 and ELAM-1, proteases, for example MMP-9, and the prothrombogenic molecule TF. Rather, by inhibiting IKK-β, a protein that affects an effective activation pathway of NF-κB specifically connected with atherosclerotic processes is inhibited.

The invention furthermore offers the advantage that, by avoiding prolonged secretion of MCP-1 from endothelial cells, the effect in avoiding immigration of monocytes is far greater than when a short-term, but greater, secretion of MCP-1 is avoided as in the case of inhibition of TNF.

In addition, the invention offers the advantage that the monocyte/macrophage flow into the vessel wall can be regulated at the level of the endothelial layer and thus before the monocytes/macrophages have penetrated into the vessel wall.

Unlike conventional treatments of atherosclerosis, according to the invention a new class of patients can be selected for therapy, for whom known therapies are ineffective or for whom known therapies are unacceptable in view of the side-effects.

A first specific sub-class of patients concerns those who are treated according to the invention with an inhibitor of IKK-β for primary prophylaxis in order to prevent a cardiovascular event, such as stroke or cardiac infarct, it being possible to select those patients and assign them to this first specific sub-class by reason of an assessment of risk factors and/or by reason of a diagnosis of an early stage of atherosclerosis.

The selection and classification of patients by reason of an assessment of risk factors can be made on the basis of an evaluation of known risk factors. Evaluation of the risk factors identified can be carried out subjectively on the basis of the general experience of the physician in charge or objectively on the basis of standardised evaluation methods. The use of a standardised evaluation method is preferred. Especially preferred is the use of the evaluation system proposed by G. Assmann et al. (Circulation 2002; 105:310-315) on the basis of the PROCAM study. In that evaluation system, standardised coefficients are assigned to the following risk factors identified for a patient, the sum of which coefficients yields a value which is correlated with a risk of the occurrence of an acute cardiovascular event in the next ten years: age, LDL-cholesterol, HDL-cholesterol, triglycerides, smoker/non-smoker, Diabetes mellitus, occurrence of MI in the family, and systolic blood pressure. Patients with a value greater than or equal to 50 can be selected and assigned to this sub-class.

The selection and classification of patients by reason of a diagnosis of an early stage of atherosclerosis can be made on the basis of an evaluation of known symptoms of the presence of atherosclerosis. Selection can be carried out especially on the basis of measurement of the average maximal intima wall thickness of the Arteria carotis intema, a thickness of more than 1.2 mm indicating atherosclerotic change. Measurement of the wall thickness is carried out with an ultrasound device that permits a suitable resolution, such as, for example, a conventional ultrasound device with a 12 MHz soundhead.

Patients who, without a cardiovascular event having previously occurred, already have lipid-rich plaques or even plaques with calcium salt deposits may also belong to this sub-class.

According to the invention, the progress of atherosclerotic changes to the vessels can be prevented by primary prophylaxis in this sub-class by administering an inhibitor of IKK-β, with the result that the risk of a cardiovascular event or an increase in the risk of a cardiovascular event is reduced.

A second specific sub-class of patients concerns those who are treated according to the invention with an inhibitor of IKK-β for secondary prophylaxis after a cardiovascular event in order to prevent further cardiovascular events, such as stroke or cardiac infarct. In that case, the patients can be selected and assigned to this second specific sub-class solely by reason of the primary event and/or, as described above, by reason of an assessment of risk factors and/or by reason of a diagnosis of atherosclerosis.

The present invention therefore relates also to a method for the prevention or treatment of atherosclerosis, which is characterised in that an inhibitor of IKK-β is administered to patients. The inhibitor can be administered by all known methods. For example, the inhibitor can be administered enterally or parenterally. On the other hand, the inhibitor can also be administered as a precursor, for example as a vector, by gene therapy. Enteral administration is preferably carried out orally. Parenteral administration may be carried out intravenously, intraarterially, intramuscularly or subcutaneously, preference being given to intravenous or intraarterial administration. It is also possible to combine enteral, parenteral and gene therapy administration of the inhibitor. In cases where the inhibitor has to be administered repeatedly over a relatively long period, an inhibitor that can be administered orally will preferably be chosen.

In a preferred embodiment, the patients concerned are those belonging to the first specific sub-class. In the case of this method for the prevention or treatment of atherosclerosis before a cardiovascular primary event, the inhibitor is administered preferably orally.

In another preferred embodiment, the patients concerned are those belonging to the second specific sub-class. In the case of this method for the prevention or treatment of atherosclerosis before a cardiovascular primary event, the inhibitor is administered preferably orally.

In another preferred embodiment, the patients concerned are those belonging to the first or the second specific sub-class, the inhibitor being administered into the endothelial cells of the vessels as a precursor by means of a vector in order to make the inhibitor available in the endothelial cells as an expression product of one or more nucleotide sequences of the vector. This approach has the advantage that repeated administration of a medicament to the patient over a relatively long period is avoided.

The invention is described in more detail below.

The inhibition of IKK-β can be effected at the DNA level by preventing the expression of the protein. Inhibition may begin at any stage of gene expression and therefore, for example, at the gene itself or at expression products, such as RNAs and proteins. Various methods for the structural alteration of genes are known from the prior art. Attention is drawn especially to the use of specific vectors. A specific use of a vector will be described in the Examples. The expression of IKK-β can also be prevented by inhibiting the transcription or translation of the IKK-β gene or of the corresponding mRNA. Examples of such inhibitors are antisense-RNAs and ribozymes.

The inhibition of IKK-β can also be effected, however, at the protein level by inhibiting the function of the expressed IKK-β protein. Inhibition at the protein level can be effected with proteins or non-proteins. Examples of inhibitor proteins are antibodies that react with epitopes of IKK-β, or proteins that interact with an active site of IKK-β and prevent phosphorylation activity. Examples of non-proteins are active substances that interact with an active site of IKK-β and prevent phosphorylation activity. For specific examples of molecular inhibitors of IKK-β reference is explicitly made to U.S. Pat. No. 5,939,302.

Preferably, the inhibitor is selective for IKK-β.

In a preferred embodiment of the present invention, the inhibitor is a compound that inhibits the expression of the gene coding for IKK-β, for example a ribozyme or an antisense-RNA. In an especially preferred embodiment, the inhibitor is an antisense-RNA that is complementary to the mRNA transcribed by the gene coding for IKK-β or to a part thereof, preferably the coding region, and that is able to bind specifically to that mRNA, thereby reducing or inhibiting the synthesis of IKK-β. In another especially preferred embodiment, the inhibitor is a ribozyme that is complementary to the mRNA transcribed by the gene coding for IKK-β or to a part thereof and that is able to bind specifically to that mRNA and cleave it, thereby reducing or inhibiting the synthesis of IKK-β. Suitable methods for the production of antisense-RNA are described in EP-B1 0 223 399 or EP-A1 0 458. Ribozymes consist of a single RNA strand and are able to cleave other RNAs intramolecularly, for example the mRNAs transcribed by the sequences coding for IKK-β. Those ribozymes must fundamentally possess two domains: (1) a catalytic domain and (2) a domain that is complementary to the target RNA and is able to bind to it, which is a pre-requisite for cleavage of the target RNA. Using procedures described in the literature as a basis, it has since become possible to construct specific ribozymes that cleave a desired RNA at a specific, pre-selected site (see, for example, Tanner et al., in: Antisense Research and Applications, CRC Press, Inc. (1993), 415-426).

In another preferred embodiment, the inhibitor is a vector.

In another preferred embodiment, the inhibitor is an antibody that binds to IKK-β, or a fragment thereof. Those antibodies may be monoclonal, polyclonal or synthetic antibodies or fragments thereof. In this context, the term “fragment” means any part of the monoclonal antibody (for example Fab, Fv or single chain Fv fragments) that has the same epitope specificity as the complete antibody. The antibodies according to the invention are preferably monoclonal antibodies. The monoclonal antibody may be an antibody originating from an animal (for example the mouse), a humanised antibody or a chimeric antibody or a fragment thereof. Chimeric antibodies resembling human antibodies or humanised antibodies have a reduced potential antigenicity, but their affinity for the target is not reduced. The production of chimeric and humanised antibodies, or of antibodies resembling human antibodies, has been described in detail (see, for example, Queen et al., Proc. Natl. Acad. Sci. USA 86 (1989), 10029, and Verhoeyan et al., Science 239 (1988), 1534). Humanised immunoglobulins have variable basic structure regions which essentially originate from a human immunoglobulin (referred to as acceptor immunoglobulin) and the complementarity of the determining regions which essentially originate from a non-human immunoglobulin (for example from the mouse) (referred to as donor immunoglobulin). The constant region(s), if present, also originate(s) essentially from a human immunoglobulin. In the case of administration to human patients, humanised (and human) antibodies offer a number of advantages over antibodies of mice or other species: (a) the human immune system ought not to recognise the basic structure or the constant region of the humanised antibody as foreign and therefore the immune response to such an injected antibody ought to be lower than towards a completely foreign mouse antibody or a partially foreign chimeric antibody; (b) since the effector region of the humanised antibody is human, it interacts better with other parts of the human immune system, and (c) injected humanised antibodies have a half-life that is substantially equivalent to that of naturally occurring human antibodies, which makes it possible to administer smaller and less frequent doses in comparison with antibodies of other species.

It is also possible to use nucleic acids that bind directly to proteins, so-called aptamers. These are identified and purified on the basis of the purified proteins from large libraries of chemically modified RNAs.

The aptamer, ribozyme, antisense-RNA and antibody inhibitors may be administered as such or alternatively may preferably be administered via gene therapy with suitable coding DNA sequences being inserted preferably into an expression vector and brought to the target tissue. The present invention accordingly includes also expression vectors containing DNA sequences coding for those inhibitors. The term “vector” refers to a virus or another suitable vehicle. The DNA sequences are functionally linked in the vector to regulatory elements that permit expression thereof in eukaryotic host cells. In addition to containing regulatory elements, such vectors contain, for example, a promoter, typically an origin of replication and specific genes that permit the phenotypic selection of a transformed host cell. Suitable regulatory sequences are additionally described in Goeddel: Gene Expression Technology: Methods in Enzymology 185, Academic Press, San Diego, Calif. (1990). Suitable expression vectors for mammalian cells include, for example, vectors originating from pMSXND, pKCR, pEFBOS, cDM8 and pCEV4 and from pcDNAI/amp, pcDNAZ/neo, pRc/CMV, pSV2gpt, pSV2neo, pSV2-dhfr, pTk2, pRSVneo, pMSG, pSVT7, pko-neo and from pHyg.

The DNA sequences described above are preferably inserted into a vector suitable for gene therapy, for example under the control of a tissue-specific promoter, and infiltrated into the cells. In a preferred embodiment, the vector containing the above-described DNA sequences is a virus, for example an adenovirus, an adeno-associated virus, a vaccinia virus or a retrovirus. Examples of suitable retroviruses are MoMuLV, HaMuSV, MuMTV, RSV or GaLV. Special preference is given to adenoviruses, especially those with E1 and/or E3 mutations (deletions) that may also have, in addition, an E4 mutation (deletion), or so-called “gutless” adenoviruses. Vectors suitable for gene therapy are also disclosed in WO 93/04701, WO 92/22635, WO 92/20316, WO 92/19749 and WO 92/06180. For the purposes of gene therapy, the DNA sequences according to the invention can also be transported to the target cells in the form of colloidal dispersions. The latter include, for example, liposomes and lipoplexes (Mannino et al., Biotechniques 6 (1988), 682).

General methods known in the specialist field can be used for the construction of expression vectors containing those DNA sequences and suitable control sequences. Those methods include, for example, in vitro recombination techniques, synthetic processes and in vivo recombination methods, as are described in current textbooks.

The above-mentioned compounds or the DNA sequences coding for them or vectors are administered, where appropriate, together with a pharmaceutically acceptable carrier.

Suitable carriers and the formulation of such medicaments are known to the person skilled in the art. Suitable carriers include, for example, phosphate-buffered saline solutions, water, emulsions, for example oil-in-water emulsions, wetting agents, sterile solutions etc. The dosage that is suitable will be determined by the physician in charge and depends upon various factors, for example the age, sex and weight of the patient, the nature and stage of the heart disease, or the mode of administration.

In another preferred embodiment, the inhibitor is an active substance selected from the following group:

Sulindac or aspirin (see Y. Yamamoto et al., J. Biol. Chem. 1999; 274 (38): 27307-14); NEMO-binding domain peptide (see May Mj. et al., Science 2000; 289; 15504); 3-[(4-methylphenyl)-sulfonyl]-2-propenenitrile and 3-[(4-methylphenyl)-sulfonyl]-2-propenenitrile (see J. W. Pierce at al., J. Biol. Chem. 1997; 272(34)).

The present invention further provides a screening method for discovering selective inhibitors of IKK-β, in which method activated endothelial cells are used to test the effectiveness of potential active substances specifically with regard to the prevention of secretion of MCP-1.

Lowered or completely inhibited enzyme activity or gene expression indicates that the test compound is effective as an inhibitor. Suitable assays are known to the person skilled in the art. This method is carried out in a cellular assay in which endothelial cells are used. The test compounds may be very diverse compounds, both naturally occurring and synthetic, organic and inorganic compounds, or polymers (for example oligopeptides, polypeptides, oligonucleotides and polynucleotides) or small molecules, antibodies, sugars, fatty acids, nucleotides and nucleotide analogues, analogues of naturally occurring structures (for example peptide imitators, nucleic acid analogues etc.) and numerous other compounds.

Furthermore, a large number of possibly useful, IKK-β-inhibiting compounds can be screened in extracts of natural products as starting material. Such extracts may originate from a large number of sources, for example from the species fungi, actinomycetes, algae, insects, protozoa, plants and bacteria. The extracts that exhibit activity can then be analysed for isolation of the active molecule. See, for example, Turner, J. Ethnopharmacol. 51 (1-3) (1996), 3943 and Suh, Anticancer Res. 15 (1995) 233-239. Fundamentally suitable assay formats for the identification of test compounds that influence the expression or activity of IKK-β are well known in the biotechnological and pharmaceutical industry, and additional assays and variations of those assays will be obvious to the person skilled in the art. Changes in the expression level of IKK-β can be studied using methods that are well known to the person skilled in the art. These include monitoring of the mRNA concentration (for example using suitable probes or primers), immunoassays as to the protein concentration, RNase protection assays, amplification assays or any other means suitable for detection and known in the specialist field.

The search for inhibitors can also be carried out on a large scale, for example by screening a very large number of candidate compounds in substance libraries, it being possible for the substance libraries to contain synthetic and/or natural molecules. At all events, the production and simultaneous screening of large banks of synthetic molecules can be performed by means of well-known methods of combinatorial chemistry, see, for example, van Breemen, Anal. Chem. 69 (1997), 2159-2164 and Lam, Anticancer Drug Des. 12 (1997), 145-167. The method according to the invention can also be greatly accelerated in the form of high throughput screening. The assays described herein can be appropriately modified for use in such a method.

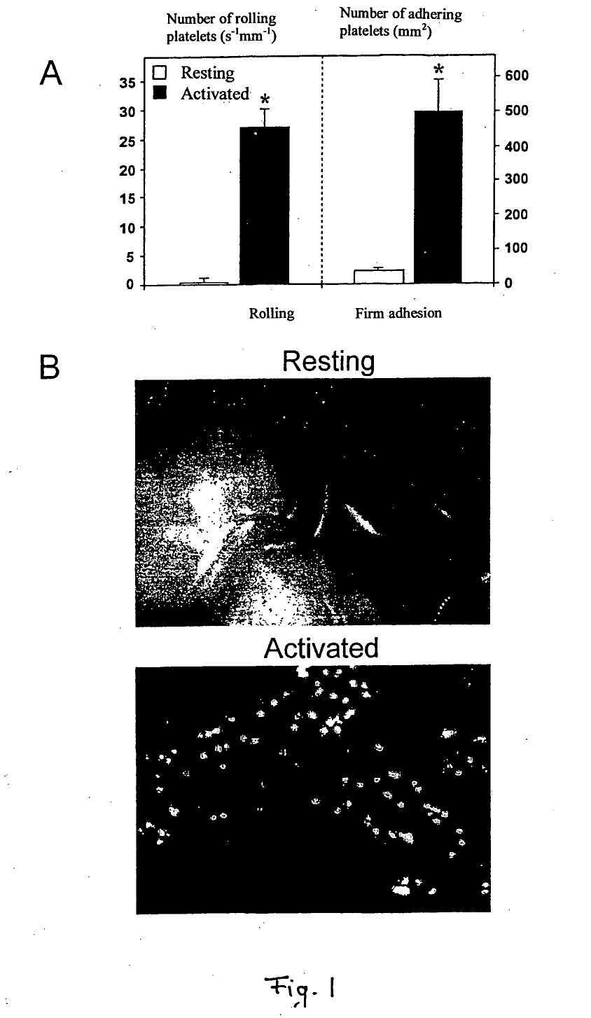

FIG. 1 shows blood platelet-endothelial cell interactions in vivo. The blood platelet-endothelial cell interactions were studied by means of intravital microscopy in arterioles of the microvasculature of mouse intestine that had been subjected to ischaemic conditions (Activated). Sham animals served as controls (Resting). FIG. 1A: blood platelets were classified according to their interactions with endothelial cells, as described in the prior art (1, 2): rolling blood platelets are given as the number of cells per second and mm of vessel diameter (rolling); adhering blood platelets are given as number per mm² of vessel surface (firm adhesion). Mean value +−SEM, n=6 test animals per group. *p<0.01 compared with sham (Dunn's method). FIG. 1B: rhodamine-6G-labelled platelets were visualised in arterioles using intravital fluorescence microscopy. Although only few blood platelets adhere to the endothelium in sham animals, adhesion of blood platelets to endothelial cells under ischaemic conditions is significantly increased. Image enlargement: ×450.

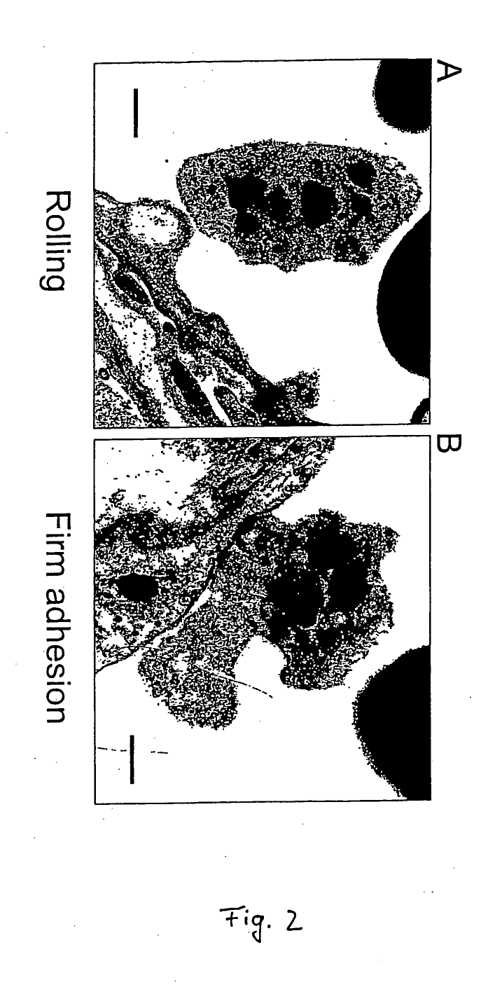

FIG. 2 shows blood platelet-endothelium adhesion on an endothelial monolayer, visualised by electron microscopy. Electron microscopy was used to show that blood platelets adhere to an intact post-ischaemic endothelial monolayer. FIG. 2A: a rolling blood platelet is shown with loose contact with the endothelium. Note the normal appearance and the normal distribution of the blood platelet granules. FIG. 2B shows a blood platelet firmly adhering to the endothelial monolayer. In regions where spreading and firm adhesion have occurred, the blood platelet has undergone significant degranulation, which indicates release of compounds originating from the platelet. Enlargement: ×20,000. Scale bars represent 0.5 μm.

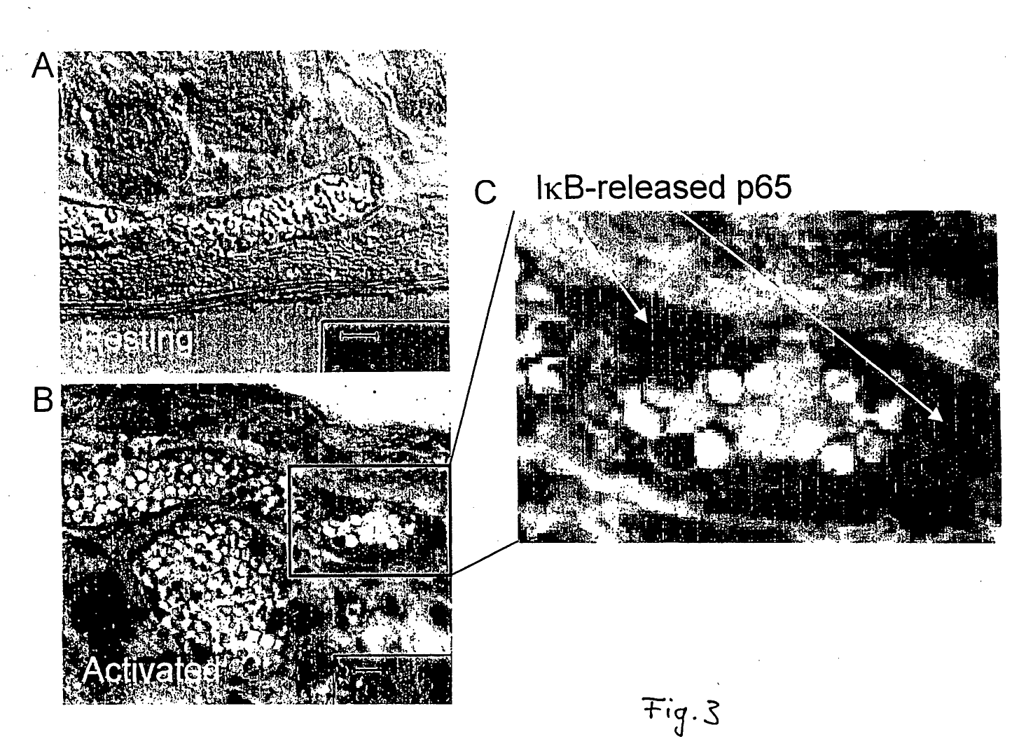

FIG. 3 shows that adhesion of blood platelets to the endothelium is related to activation of NF-κB. Cryostat sections of visceral samples were incubated with a monoclonal antibody (mAb) to p65 released by IκB (=activated NF-κB), followed by staining by means of the peroxidase-antiperoxidase technique. p65 immune reactivity is indicated by the red reaction product (which appears dark in the Figure). FIG. 3A: in control animals (Resting), an immune reactivity of p65 in the vascular endothelium was as good as non-existent. FIG. 3B: in experiments with increased blood platelet-endothelium adhesion (Activated), p65 staining in arterioles and the surrounding tissue was to be found on an increased scale. FIG. 3C: in regions of massive blood platelet accumulation, which is shown in an enlargement of a portion of FIG. 3B, p65 released by IκB was present predominantly in the endothelial monolayer (arrows). Enlargement: ×40 in FIGS. 3A and 3B and ×100 in FIG. 3C. Scale bars represent 10 μm.

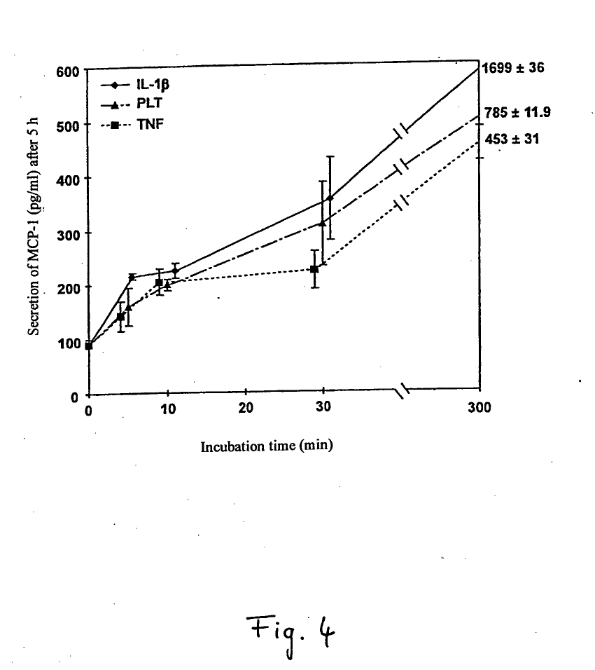

FIG. 4 shows the effect of transient interaction of activated blood platelets with HUVEC on the secretion of MCP-1. HUVEC were treated for from 5 to 300 minutes either with or without α-thrombin-activated blood platelets (PLT; 2×10⁸/ml), IL-1β (100 pg/ml) or TNF (1.67 ng/ml). The blood platelets or cytokines were then removed by washing and new culture medium was added. The cells were incubated for a total of 5 hours. The supematant was then removed by suction and used in an ELISA for measuring MCP-1. The curves show the mean value +−standard deviation from four independent experiments, each of which were carried out three times.

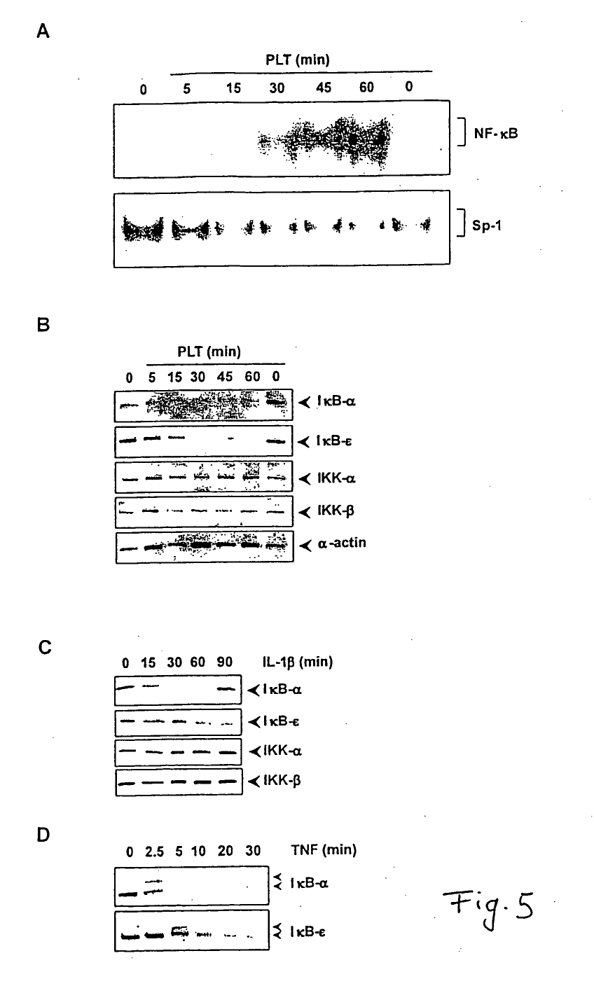

FIG. 5 shows the stimulation-dependent NF-κB activation and the IκB degradation in HUVEC. FIG. 5A: nuclear extracts from HUVEC stimulated with blood platelets (PLT; 2×10⁸/ml) were examined by EMSA for NF-κB or Sp-1 binding, which is shown in brackets. FIG. 5B: cytosol extracts from cells stimulated with blood platelets were analysed by means of western blots. The proteins IκB-α, IκB-ε, IKK-α and IKK-β are indicated by arrow heads. α-Actin was used as a loading control. FIG. 5C: cytosol extracts from cells stimulated with IL-β (100 pg/ml) were examined by means of western blots. FIG. 5D: cells were incubated with TNF (1.67 ng/ml) and cytosol extracts were examined by means of western blots. The top arrow indicates the phosphorylated forms of IκB-α or IκB-ε. A representative blot is shown for all cases (n=3).

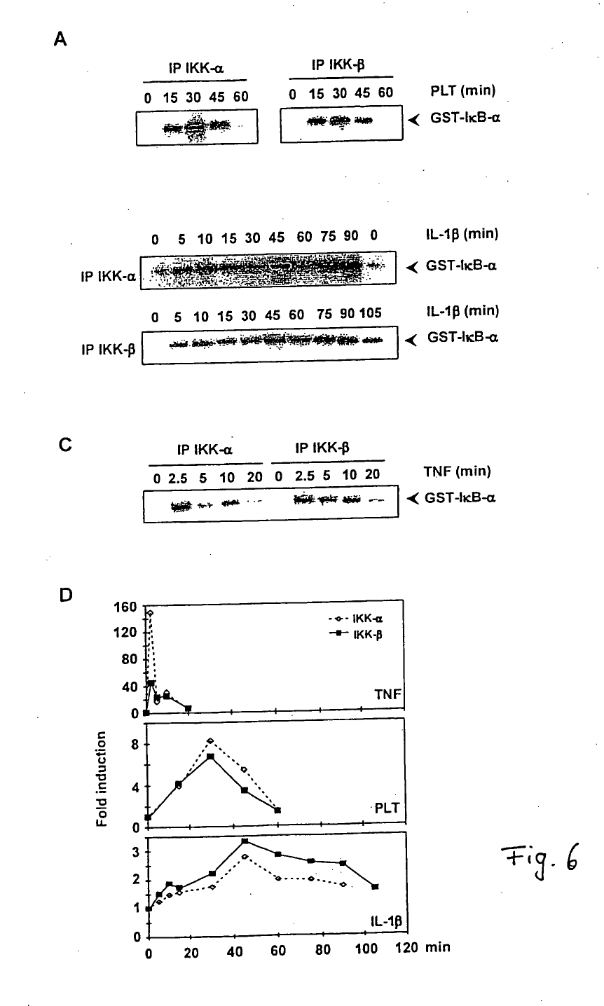

FIG. 6 shows the differential IKK complex activation in endothelial cells. Cells were incubated as indicated and cytosol extracts were made. Immunoprecipitation (IP) with antikinase antibodies as indicated was followed by a kinase assay using the substrate GST.IκB-α in the presence of ³²P-ATP. A time-dependent phosphorylation of the substrate is shown in each case after stimulation of HUVEC with blood platelets (PLT; 2×10⁸/ml) (FIG. 6A), IL-1β (100 pg/ml) (FIG. 6B) or TNF (1.67 ng/ml) (FIG. 6C). A representative experiment is shown for each case (n=3). FIG. 6D: the kinase assays were scanned and analysed densitometrically. The diagram shows the factors of induction of IKK activity compared with the control after stimulation.

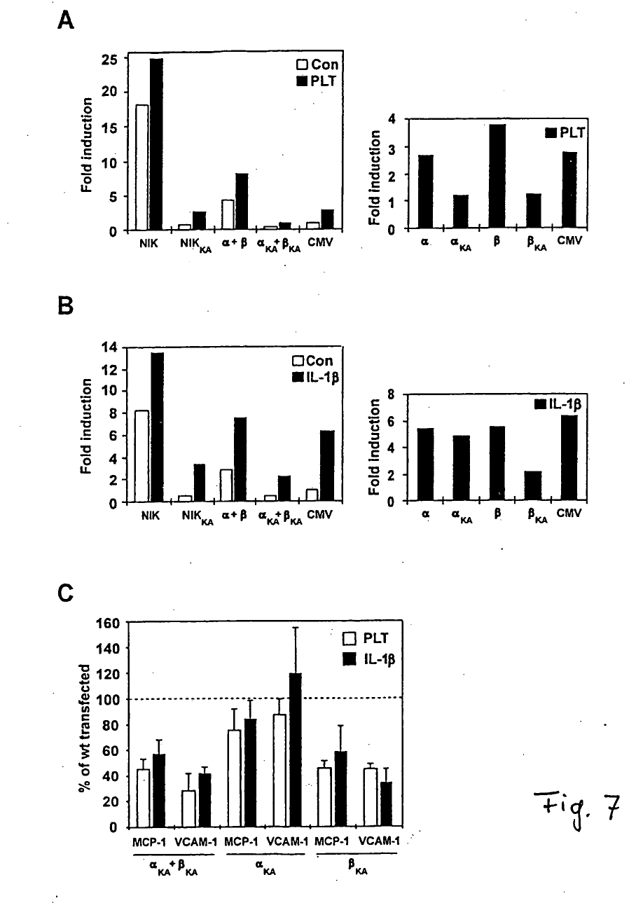

FIG. 7 shows that κB-dependent and MCP-1/VCAM-1-promoter-dependent transcription is inhibited by overexpression of dominant-negative IKK complex components. FIG. 7A and FIG. 7B: ECV304 cells were cotransformed with 3kB.luci, pRLtk and kinase-overexpression plasmids (IKK-α, -β, or NIK, wild-type or mutated, or an empty vector, CVM) in single or double transformations, as indicated. After incubation overnight, the cells were stimulated for 60 minutes with either activated blood platelets (PLT; 108/ml) (FIG. 7A) or IL-1β (100 pg/ml) (FIG. 7B). After a further 15 hours, the cells were lysed and the glow worm and Renilla RLU was measured. The results are shown as multiples of the induction compared with those obtained with unstimulated CMV-transformed cells. The bars on the left show the effect of either wild-type (wt) or mutated NIK and of IKK-α and -β on the stimulus-induced κB-dependent transcription. Representative experiments are shown (n=3).

Con: unstimulated cells. FIG. 7C: cells were transformed as indicated with either an MCP-1- or a VCAM-1-promoter-dependent luciferase reporter plasmid together with pRLtk and IKK by means of expression plasmids. After stimulation with blood platelets or IL-1β, glow worm and Renilla RLU was measured. The results are shown as a percentage of the inhibition obtained relative to the cells transformed with the wild type (wt) which are indicated with the dotted line at 100%. The bars show the mean value ±standard deviation from three independent experiments. α, IKK-α; β, IKK-β: KA, the kinase-active site is mutated.

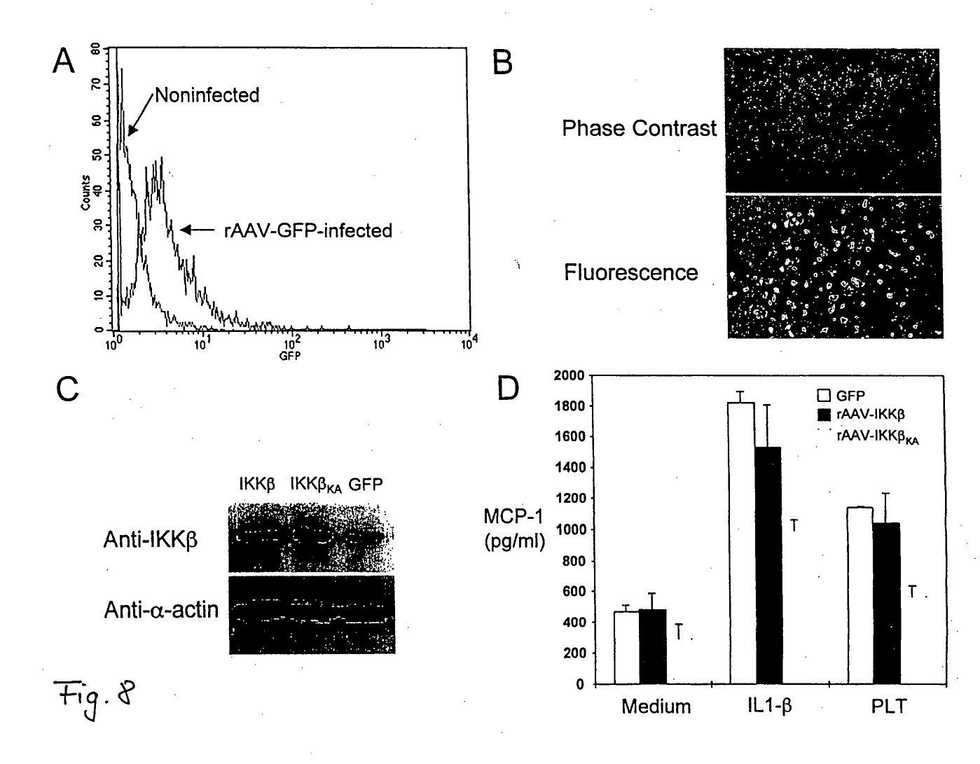

FIG. 8 shows that the rAAV-mediated dominant-negative IKK-β transfer inhibits blood-platelet-induced and IL-1β induced endothelial MCP-1 secretion. Subconfluent HUVEC were left untreated or were transformed with control rAAV-GFP, wild-type rAAV-IKK-β or dominant-negative mutants rMV-IKK-β_{KA}. 48 hours after transformation, the cells were left untreated or were stimulated for 60 minutes with activated blood platelets (PLT; 2×10⁸/ml) or IL-1β (100 pg/ml), washed and further incubated as indicated. FIG. 8A: the cells were examined for GFP expression using flow cytometry in order to determine transformation efficiency. The curve marked “noninfected” indicates noninfected cells and the curve marked “rAAV-GFP-infected” indicates cells that were treated with rAAV-GFP. Approximately 50% of the HUVEC are infected with rMV-GFP. FIG. 8B: GFP expression was analysed by fluorescence microscopy. Top image: phase contrast of the HUVEC monolayer; bottom image: corresponding green-fluorescence (bright spots) micrography. FIG. 8C: cytosol extracts of HUVEC transformed with rMV-IKK-β, rMV-IKK-β_{KA} or rAAV-GFP were examined by means of western blots for endogenous (GFP) and overexpressed IKK-β protein. α-Actin was used as a loading control. FIG. 8D: 4 hours after washing, soluble MCP-1 in the supernatant of the cells was quantitatively determined by means of ELISA. The data are shown as mean values ±standard deviation from in each case three experiments carried out in duplicate.

The invention will be explained in more detail below by means of specific Examples.

### EXAMPLES

### Materials and Methods

Animals and Intravital Fluorescence Microscopy

Female Balb/c mice (Charles River, Suzfeld) aged from 5 to 7 weeks (10 test animals, 10 platelet donors) were used. The surgical procedure is prior art (1, 2). Specifically, a segment of the jejunum was brought to the outside and subjected to a 60-minute normothermic ischaemia. Blood platelets of the mice were isolated from whole blood and labelled with rhodamine-6G. Blood platelet-endothelial cell interactions in the post-ischaemic microvasculature were examined in submucosal arterioles by intravital microscopy before and 60 minutes after the ischaemia. Five non-overlapping regions of interest were selected at random and a quantitative determination of platelet-endothelial cell interactions was carried out. Those interactions were classified as rolling adhesion or firm adhesion of platelets.

Electron Microscopy and Immunohistochemical Studies

Samples from the intestinal segments were taken before and 60 minutes after the ischaemia. For the purpose of electron microscopy, the tissue was fixed, and processed as described in the prior art (2). Ultra-thin regions were cut and stained with uranyl acetate and lead citrate and examined under a Zeiss EM 900 transmission electron microscope (Zeiss, Oberkochen) operating at 80 kV. For the purpose of immunostaining, the biopsies were introduced into an OCT compound (Tissue-Tek; Miles Inc., Elkhart, Ind., USA) and immediately frozen with liquid nitrogen. In order to visualise activated NF-κB, acetone-fixed cryostat sections (6 μm) were incubated with biotin-conjugated mouse-anti-human α-p65mAb (Roche Diagnostics, Mannheim), which selectively reacts with p65 only in the absence of IκB and therefore recognises the activated form of NF-κB, and which cross-reacts with the corresponding mouse antigen (3). After incubation with the primary antibody, the sections were stained with peroxidase immunohistochemistry kits (Vectastain, Camon, Wiesbaden). Endogenous peroxidase activity was blocked with methanol/H₂O₂ for 10 minutes at room temperature.

Cell Culture

Primary cultures from human umbilical vein endothelial cells (HUVEC) were obtained from umbilical veins by collagenase treatment as described in (4). Cells from 4-6 preparations were combined and cultivated on 24-well culture plates in complete endothelial cell growth medium (PromoCell, Heidelberg) comprising 2% FCS, 1 μg/ml hydrocortisone, 0.1 ng/ml hEGF, 1.0 ng/ml FGF, 50 μg/ml gentamycin and 2.5 μg/ml amphotericin B.

The human endothelial cell line ECV304 (American Tissue Culture Collection, Rockville, Md., USA) was cultivated in M199 enriched with 10% FCS, 1% sodium pyruvate, 1 mM L-glutamine, 100 U/ml penicillin, 100 μg/ml streptomycin (Biochrom, Berlin). To eliminate endotoxin contamination, all crystalloid solutions were ultrafiltered (U 200, Gambro, Hechingen), and mother solutions of proteins were decontaminated with polymyxin columns (Pierce, Rockford, Ill., USA). In order to rule out endotoxin contamination, all cell suspensions were evaluated after every experiment by a chromogenic Limulus amoebocyte lysate assay (Schulz, Heidelberg).

Determination of MCP-1

The concentrations of MCP-1 protein were determined in HUVEC supernatants by ELISA (Quantikine R&D, Wiesbaden-Nordenstadt).

Co-cultivation of Endothelial Monolayers with Blood Platelets

Blood platelets were isolated from acid citrate dextrose anticoagulated whole blood collected from healthy individuals who were non-smokers and who were not under the influence of any medicaments known to affect blood platelet function. The blood platelets were washed and resuspended in Tyrode's solution/HEPES buffer (HEPES 2.5 mM, NaCl 150 mM, NaHCO₃ 12 mM, KCl 2.5 mM, MgCl₂ 1 mM, CaCl₂ 2 mM, D-glycose 5.5 mM, 1 mg/ml BSA, pH 7.4) in order to provide a blood platelet count of 4×10³/ml as described in (5). Blood platelets were pre-stimulated for 20 minutes with 2 U/ml α-thrombin (Sigma, Deisenhofen), followed by thrombin antagonisation with 5 U/ml hirudin (Roche Diagnostics). Blood platelet activation was verified by ascertaining the P-selectin surface expression and the receptor of activated fibrinogen (LIBS-1, Dr. Mark Ginsberg, Scripps Research Institute, La Jolla, Calif., USA). 1 ml of the blood platelet suspension was added to 1 ml of complete medium (final blood platelet count 2×10⁸/ml) and transferred to wells containing confluent HUVEC monolayers on 6-well plates. The cells were left untreated or were incubated with blood platelets, as indicated, at 37° C.

Finally, the blood platelets were removed by several careful washing steps, and culture medium was added to the cells until the complete incubation time had elapsed. In preliminary studies using a blood-platelet-specific anti-CD42b mAb, it was made sure by microscopy and flow-cytometry that all blood platelets had been removed from the endothelial monolayer by the washing process.

Electrophoretic Mobility Shift Assay (EMSA=Gel Retardation Assay)

Nuclear extracts of the cells were made and analysed as described in the prior art (6). The prototypic immunoglobulin κ-chain oligonucleotide was used as a probe and labelled with the Klenow fragment of DNA polymerase I (Roche Diagnostics). Nuclear proteins were incubated with probes radio-labelled by [α-³²P]dCTP (NEN Life Science Products, Brussels, Belgium) for 30 minutes at room temperature in binding buffer (6). Samples were run in 0.25× TBE on non-denaturing 4% polyacrylamide gels. Binding of the transcription factor Sp-1 was analysed using a consensus oligonucleotide (Promega, Heidelberg) that had been labelled with [γ-³²P]ATP (NEN Life Science Products) and T4 polynucleotide kinase (Roche Diagnostics). The gels were dried and analysed by autoradiography.

PAGE and Western Blot Analysis

Cytosol extracts were isolated, and electrophoresis and blotting were carried out as described in the prior art (6, 7). After transfer, the membranes were incubated with antibodies to IκB-α (Santa Cruz Biotechnology, Heidelberg), IκB-ε (Prof. N. Rice, NIH, Frederick, Md.), IKK-α, IKK-β (Santa Cruz Biotechnology) or α-actin (Sigma). The proteins were visualised on X-ray film using a western blot chemiluminescence reagent (NEN Life Science Products). Protein sizes were confirmed by means of molecular weight standards (Amersham Pharmacia Biotech, Braunschweig, Bio-Rad, Munich).

Immunoprecipitation and Kinase Assay

Cytosol extracts were subjected to immunoprecipitation (IP) (7) in TNT buffer containing 1 mM dithiothreitol, 0.5 μM 4-(2-aminoethyl)-benzenesulfonyl fluoride (AEBSF) and 0.75 μg/ml each of leupeptin, antipain, aprotinin, pepstatin A and chymostatin (Sigma). Non-specific binding was blocked by incubation with 1 μg of normal hare-IgG (Santa Cruz Biotechnology) and 25 μl of 6% protein A-agarose (Roche-Diagnostics) for 30 minutes at 4° C., followed by immunoprecipitation for 2 hours at 4° C. with 1 μg of a suitable anti-kinase antibody (Santa Cruz Biotechnology). After washing three times with TNT and kinase buffer each time (20 mM HEPES, pH 8.0; 10 mM MgCl₂, 100 μM Na₃VO₄; 20 mM β-glycerophosphate; 50 mM NaCl; 2 mM dithiothreitol; 0.5 μM AEBSF; 0.75 μg/ml each of leupeptin, antipain, aprotinin, pepstatin A, chymostatin), the precipitated proteins were analysed by a kinase assay. The reaction was carried out in kinase buffer for 30 minutes at 30° C. in the presence of 5 μCi of [γ-³²P]ATP (NEN Life Science Products) and GST-IκB-α (Santa Cruz Biotechnology). Proteins were analysed with PAGE and visualised by means of autoradiography.

Transfection of Endothelial Cells

The following reporter plasmids were used in transfection studies:

3xκB-luci, a luciferase reporter plasmid of the glow worm that contains 3 copies of a prototypic κB site (7);

VCAM-1.luci, comprising 1811 base pairs of the VCAM-1 promoter region (Dr. Nigel Mackmann, Scripps Research Institute, La Jolla, Calif.); and

MCT-1.luci, containing the proximal promoter and distal enhancer regions of the human MCP-1 gene (8, 9) (Dr. Teizo Yoshimura, NIH, Frederick, Md.).

The following overexpression plasmids were used:

IKK-α,

IKK-β and

NIK.

(wild-type and mutated, kinase-inactive forms, Dr. Mike Rothe, Tularik Inc., South San Francisco, Calif.).

Empty RcCMV (Invitrogen, Groningen, Holland) was used as a negative control. These plasmids, either individually or in combination, were transiently cotransformed with a constitutively active Renilla luciferase control plasmid, PRLtk (Promega), in endothelial cells as described in the prior art (10). After 24 hours, the cells were stimulated for 60 minutes with either IL-1β or blood platelets, followed by cultivation for a further 15 hours. After stimulation, the cells were lysed and the luciferase activity was determined using the dual luciferase reporter assay system (Promega). The results are expressed as a ratio of the relative light units (RLU) of fireflies and Renilla luciferase.

Construction and Purification of a Recombinant Adeno-associated Virus

The plasmid pTR-UF5 was provided by Dr. Muzyczka (Gene Therapy Center, Gainsville, Fla.) (11, 12) and the plasmid pDG was provided by Dr. Kleinschmidt (Deutsches Krebsforschungszentrum, Heidelberg, Germany) (11-13). A recombinant, replication-deficient adeno-associated virus (rAAV) containing the green4luorescent protein (rAAV-GFP), human wild-type IKK-1β (rMV-IKK-β) or mutated IKK-β_{KA} (rAAV-IKK-β_{KA}) was constructed as follows: the coding sequence of the wild-type or the mutated IKK-β was inserted into the polylinker sequence of the pTR-UF5 vector. The resulting plasmids were then cotransformed with the pDG plasmid in subconfluent HEK 293 cells and using a modified calcium phosphate co-precipitation method, and a production of rAAV was carried out as described in the prior art (13, 14). The titres of infectious virus particles (IVP) were assessed by infection of proliferating HeLa cells.

Transduction of Endothelial Cells

HUVEC were plated out onto 96-well plates, at 10⁴ cells per well, in 200 μl of culture medium. At subconfluence, approximately from 16 to 24 hours later, from 1 to 3×10³ IVP of rAAV were added to each cell. The media were changed 16 hours after the addition of rMV, and transduced cells were cultivated for a further 48 hours. The cells were then stimulated with IL-1β or α-thrombin-activated blood platelets for 60 minutes, and secretion of MCP-1 was evaluated 4 hours later. Efficiency of endothelial rAAV-GFP transduction was evaluated by flow cytometry, fluorescence microscopy and western blot.

Results

Blood Platelet Interaction in vivo

The mouse model of ischaemia which was used here allows the differentiation of various types of blood platelet adhesion which was visualised by intravital microscopy.

In the in vivo physiological state, circulating blood platelets seldom interacted with the microvascular endothelium (FIG. 1A and B, Resting). Few blood platelets were observed rolling along the endothelial cell lining of arterioles (<1/s/ml). At the same time, only 38±16 blood platelets that firmly adhered were found per mm² of endothelial cell surface.

In contrast, in cases where similar measurements were carried out in arterioles under ischaemic conditions (60 min ischaemia), a dramatically increased blood platelet-endothelial cell adhesion was found (FIG. 1A and B, Activated). An increase of approximately 160-fold was found in the case of rolling interaction and an increase of 13-fold in firm platelet adhesion to ischaemic endothelium (P<0.01).

Firm Adhesion of Blood Platelets to the Endothelium Induces Granule Secretion

In order to study morphological changes that occur in blood platelets when they adhere to vascular endothelium in vivo, electron microscopy studies were carried out. It was found that individual blood platelets adhere to endothelial cells also in the absence of aggregates, without defects being perceptible in the endothelial cell layer (FIG. 2). These blood platelets loosely adhering to endothelial cells (rolling blood platelets) exhibited the typical pattern of complete granules that are not yet degranulated (FIG. 2a). In contrast to this, blood platelets that are spread over the endothelial cell surface (firm adhesion) are characterised by a strong secretion of their granules, as is evidenced by empty organelles found in those regions (FIG. 2B).

NF-κB is Activated in the Microvasculature in Regions of Blood Platelet Adhesion

It is known from in vitro studies that interaction of activated blood platelets with endothelial cells (permanent co-culture) leads to gene expression of chemokines and adhesion molecules via activation of NF-κB. In order to study the activation of that transcription factor in regions of increased blood platelet-endothelium adhesion in the microvasculature, immunohistochemistry was carried out on cryostat sections using an mAb to the NF-κB p65 sub-unit released by IκB. In sham mice, activation of NF-κB is almost completely absent (FIG. 3A, Resting). Under conditions of increased blood platelet-endothelium interaction, an increased immunoreactivity of activated NF-κB, shown by p65-labelling, was observed in the microvasculature (FIG. 3B, Activated) and predominantly in the endothelial monolayer adjacent to the blood platelet aggregates (FIG. 3C). This indicates that an increased blood platelet adhesion to the endothelium leads to activation of NF-κB in vivo.

Transient Incubation of HUVEC with Activated Blood Platelets Induces Secretion of MCP-1

Since significant quantities of activated NF-κB are observed in regions exhibiting blood platelet adhesion, the effects of activated blood platelets on cultivated HUVEC were studied. First, the time course of the blood-platelet-induced endothelial secretion of the chemokine MCP-1 regulated by NF-κB (8, 9) was studied in vitro. Confluent monolayers of HUVEC were left untreated or were incubated for from 5 to 300 minutes with α-thrombin-activated blood platelets, 100 pg/ml IL-1β or 1.67 ng/ml TNF. Blood platelets or cytokines were then removed from the endothelial monolayer, and the quantity of MCP-1 in the supernatant was determined after a further four hours' incubation (short-term co-culture).

Transient incubation of HUVEC with activated blood platelets for 30 minutes produced a great increase in MCP-1 secretion, which is comparable to IL-1β-stimulation (FIG. 4), the effect of TNF being somewhat weaker. Incubation of HUVEC with resting blood platelets did not induce any significant increase in MCP-1 secretion.

Endothelial NF-κB-activation and IκB-α/ε Proteolysis Pattern After Incubation with Blood Platelets

In order to shed more light on the mechanism by which activated blood platelets induce the expression of NF-κB target gene products, such as MCP-1, time course experiments were carried out in order to observe an NF-κB activation which was determined by EMSA. Activated blood platelets induced a strong and time-dependent activation of NF-κB in HUVEC, maximum effects being observed after 60 minutes (FIG. 5A). In the same nuclear extracts, the binding of proteins to Sp-1 oligonucleotides, which act as a loading control (FIG. 5A), was also studied.

The time frame and the scope of the blood-platelet-induced proteolysis of the IκB inhibitor protein was then studied. Incubation of HUVEC with activated blood platelets and with IL-1β resulted in significant proteolysis of IκB-α and IκB-ε (FIG. 5B and C). Although the proteolysis pattern of the IκB proteins following both stimuli were similar, differences were observed: IκB-α was degraded somewhat more rapidly after being acted upon by IL-1β, beginning 15 minutes after incubation, than in the case of a blood platelet stimulation where degradation was observed after approximately 30 minutes. Conversely, blood platelets induced earlier IκB-ε proteolysis than IL-1β (significant effect: 30 as opposed to 60 minutes). In sharp contrast to this, TNF induced a far more rapid degradation of both IκB proteins in HUVEC (FIG. 5D).

Differential IKK Complex Activation in Endothelial Cells After Stimulation with Blood Platelets, IL-1β or TNF

Before their degradation, IκB proteins are phosphorylated, this process being brought about by the IKK complex. In order to characterise the effect of blood platelets on this kinase system, in vitro kinase assays were used. HUVEC were stimulated with activated blood platelets, IL-1β or TNF for the periods of time indicated before cytosol extracts were made. Immunoprecipitation was carried out with either IKK-α or IKK-β antibodies, and the kinase activity was determined by measuring the phosphorylation status of GST-IκB-α. Incubation with blood platelets induced a marked activation of the IKK complex with maximum phosphorylation levels at 30 minutes, which rapidly fell to base line values within 60 minutes (FIG. 6A and D). Similar results were obtained when IL-1β was added to HUVEC, although differences were observed with an activation peak at 45 minutes which was maintained for at least 90 minutes (FIG. 6B and D). When TNF was used to stimulate HUVEC, activation of IKK rapidly commenced within 2.5 minutes and returned to low values within 20 minutes (FIG. 6C and D). IKK-α and -β levels remained constant under those conditions (FIG. 5B and C). Similar results were obtained using ECV 304 cells.

IKK-β and NIK are Involved in Blood-platelet-induced NF-κB Activation

In order to study the role of IKK and NIK proteins in signal cascades relating to NF-κB, ECV 304 were transformed, prior to stimulation with blood platelets or IL-1β, with overexpression vectors for wild-type or mutated Flag-labelled kinases together with a κB luciferase reporter plasmid. A western blot using an anti-Flag antibody confirmed that the particular proteins were overexpressed in the system concerned and that the control vector, without insertion, did not exhibit any non-specific Flag labelling. Additional experiments showed that Flag-labelled precipitated proteins were kinase-active, the mutated forms exhibiting no activity. When NIK was overexpressed, a dramatic induction of the 3×κB.luci signal was observed, compared with the CMV control, which was further increased by incubation of the cells with activated blood platelets or IL-1β (FIG. 7A and B, left-hand columns). Transfection of mutated NIK caused a dramatic lessening of this effect with both the wild-type-induced signal and the additional increase by further stimulation. Cotransforming wild-type IKK-α and -β together also had a direct effect on the 3κB.luci plasmid, which was made significantly stronger by stimulation with either blood platelets or IL-1β (FIG. 7A and B, left-hand column). Overexpression of the mutated forms of IKK-α and -β together inhibited both the direct and the stimulus-induced effects, the blood-platelet-induced signal disappearing almost completely, while a weaker effect was observed for IL-1β. When the kinases were expressed individually instead of in combination, a significant inhibition with IKK-β_{KA} was found as a consequence of incubation with blood platelets or IL-1β (FIG. 7A and B, right-hand columns). IKK-α_{KA} exhibited an inhibitory effect only for blood platelets, but not for IL-1β-stimulated cells.

The effect of overexpression of mutated IKK-α and -β on the blood-platelet-induced or the IL-1β-induced promoter-dependent transcription was also studied. ECV304 were transformed with overexpression vectors for wild-type or mutated kinases together with luciferase reporter plasmids containing MCP-1 or VCAM-1 κB-carrying promoter fragments. In that case, only IKK-β_{KA}, but not IKK-α_{KA}, produced a significant reduction in blood-platelet-induced or IL-1β-induced transcription under regulation of those promoters (FIG. 7C).

IKK-β_{KA} Overexpression Inhibits Blood-platelet-induced Secretion of MCP-1 in rAAV-transduced HUVEC

In order to demonstrate the effect of IKK-β_{KA} overexpression at the protein level, wild-type or mutated IKK-β sequences were cloned in a recombinant adeno-associated virus system (13, 14). The studies were originally carried out in order to test whether rAAV are able to transduce primary HUVEC cultures. Subconfluent endothelial monolayers were incubated with rAAV-GFP for 16 hours and, after a further 48 hours in cell culture, the expression of GFP in infected HUVEC was analysed by flow cytometry and fluorescence microscopy (FIG. 8A and B). It was found that, under the conditions used, there was a substantial transduction of HUVEC with an efficiency of approximately 50%. This is in advantageous contrast to the transfection rate of approximately from 5 to 10% found in HUVEC when conventional transfection methods are used. The overexpression of IKK-β by the rAAV system was confirmed using a western blot analysis (FIG. 8C). In order to study the effects of IKK-β and IKK-β_{KA} overexpression, HUVEC were transduced with rAAV that codes for those proteins or with the control rAAV-GFP. After the initial incubation, which was followed by further cultivation for 48 hours, the transduced cells were treated for 60 minutes with activated blood platelets or IL-1β, and secretion of MCP-1 was determined after 4 hours. Cells that had been transduced with rAAV-IKK-β_{KA} exhibited a significant decrease in blood-platelet-induced or IL-1β-induced MCP-1 secretion compared with rAAV-GFP or rAAV-IKK-β-treated cells (FIG. 8D). These results show that the rAAV system and the high transfection efficiency which is thereby obtained permits, by determination of the protein concentration, the direct proof that the dominant-negative mutant IKK-β_{KA} strongly inhibits the expression of the endothelial inflammation gene MCP-1 which is induced by transient interaction with activated blood platelets or IL-1β.

Literature:

1. Massberg et al. (1998) Blood 92, 507-512.

2. Massberg et al. (1999) Blood 94, 3829-3838.

3. Brand et al. (1996) J Clin Invest 97, 1715-1722.

4. Jaffe et al. (1973) J Clin Invest 52, 2745-2752.

5. Dickfeld et al. (2001) Cardiovasc Res 49, 189-199.

6. Page et al. (1999) J Biol Chem 274, 11611-11618.

7. Fischer et al. (1999) J Biol Chem 274, 24625-24632.

8. Ueda et al. (1997) J Biol Chem 272, 31 092-31 099.

9. Martin et al. (1997) Eur J Immunol 27, 1091-1097.

10. Gawaz et al. (1998) Circulation 98, 1164-1171.

11. Zolotukhin et al. (1999) Gene Ther 6, 973-985.

12. Grimm and Kleinschmidt (1999) Hum Gene Ther 10, 2445-2450.

13. Hermens et al. (1999) Hum Gene Ther 10, 1885-1891.

14. Hauswirth et al. (2000) Methods Enzymol 316, 743-761.

1-6. (canceled).

7. A screening method for identifying inhibitors of IKK-β in endothelial cells, wherein the method comprises contacting a potential inhibitor with activated endothelial cells and determining secretion of MCP-1 from the endothelial cells.

8. The screening method according to claim 7, wherein the endothelial cells are activated with activated blood platelets.

9. Use of an inhibitor of IKK-β, which inhibitor is selected from the group consisting of sulindac, NEMO-binding domain protein, 3-[(4-methylphenyl)-sulfonyl]-2-propenenitrile and 3-[(4-t-butylphenyl)-sulfonyl]-2-propenenitrile for the preparation of a medicament for
(a) primary prophylaxis of patients having a PROCAM-value greater than or equal to 50; or
(b) secondary prophylaxis after a cardiovascular event.
